(19) United States
(12) Patent Application Publication
(10) Pub. No.: US 2013/0317378 A1
(43) Pub. Date: Nov. 28, 2013

(54) Assessment of Pulmonary Blood Flow and Systemic Blood Flow in a Single Ventricle Patient

(71) Applicants: Nikolai M. Krivitski, Ithaca, NY (US); Victor Kislukhin, Ithaca, NY (US); Naveen Thuramalla, Ithaca, NY (US); Anton Kriksunov, Ithaca, NY (US)

(72) Inventors: Nikolai M. Krivitski, Ithaca, NY (US); Victor Kislukhin, Ithaca, NY (US); Naveen Thuramalla, Ithaca, NY (US); Anton Kriksunov, Ithaca, NY (US)

(21) Appl. No.: 13/481,339

(22) Filed: May 25, 2012

(51) Int. Cl. A61B 5/029

(52) U.S. Cl. 600/526

(57) ABSTRACT

A system and accompanying method is provided for assessing a ratio of pulmonary to systemic blood flow in patients with a single-ventricle physiology (SVP). A compound dilution curve is recorded in an arterial vessel downstream of the pulmonary artery. A first component of the compound dilution curve is identified, wherein the first component is attributable to the indicator after passing through the single ventricle heart and directly into the arterial vessel A second component of the compound dilution curve is identified, wherein the second component is attributable to the indicator after passing from the single ventricle heart to the lungs, through the single ventricle heart and then to the arterial vessel downstream of the pulmonary artery. Based on the identified components, the pulmonary flow and systemic flow are assessed as corresponding to the identified first component and second component of the compound dilution curve.

### CROSS-REFERENCE TO RELATED APPLICATIONS

Not applicable.

### STATEMENT REGARDING FEDERALLY SPONSORED RESEARCH OR DEVELOPMENT

Not applicable.

### REFERENCE TO A “SEQUENCE LISTING”

Not applicable.

### BACKGROUND OF THE INVENTION

1. Field of the Invention

The present disclosure relates to assessing pulmonary and systemic blood flow in patients with a single ventricle physiology (SVP), and more particularly to determining a ratio of pulmonary blood flow (Qₚ) to systemic blood flow (Qₛ) in patients with SVP.

2. Description of the Related Art

Congenital heart defects are the most common type of birth defects and are responsible for more deaths in the first year of life than any other birth defects. Recently, new surgical methods have been developed to extend the life of patients born with single ventricles or SVP.

One such method for Hypoplastic left heart syndrome is called the Norwood procedure. After this type of surgery, patients have a single-ventricle that pumps blood into the aorta, and then a part of this blood is redirected into the pulmonary artery (PA). In another type of surgery for this patient population, blood from the single ventricle is simultaneously directed into the aorta and the PA.

The blood flow into the PA is termed Qₚ, and the blood flow into the “systemic” tissue (brain, liver, kidneys, myocardium etc.) is termed Qₛ. A Qₚ/Qₛ ratio significantly lower than 1 can lead to hypoxia and brain injury, cyanosis and other related problems. On the other hand, a Qₚ/Qₛ value significantly higher than 1 can result in insufficient tissue perfusion, pulmonary over-circulation as well as lung edema.

The treatment strategy primarily depends on the correct assessment of the Qₚ/Qₛ ratio. Therapeutically, depending on the Qₚ/Qₛ value, the clinician can decide whether to change the systemic vascular resistance and pulmonary vascular resistance using powerful medications in pediatric patients.

Current methods to assess the Qₚ/Qₛ value such as Oximetric Techniques (Fick Method), requires drawing blood samples from multiple sites including the pulmonary artery. This approach requires placement of highly invasive catheters including one in the PA and drawing multiple blood samples. This method is only feasible during a Cathlab investigation, but not at the bed side of the patient in an intensive care unit (ICU) where assessment is often most needed for treatment. Current bed side methods also taking and measuring multiple blood samples in accordance with numerous assumptions, but often lead to less than accurate assessments.

However, there are significant risks associated with incorrect or inaccurate assessments of Qₚ/Qₛ. Such assessments can results in moving the Qₚ/Qₛ in the wrong direction which can dramatically worsen the condition of patient, including sudden death. Similarly, an inaccurate assessment can permit an unknowingly movement of the Qₚ/Qₛ value, thus also leading to dramatic worsening of a patient's situation.

Therefore, the need exists for a simpler assessment of Qₚ/Qₛ. It is believed the timely and accurate quantitative assessment of Qₚ/Qₛ permits increased success in pharmacologic, ventilator, fluid therapy, or in-time surgical intervention in SVP.

### BRIEF SUMMARY OF THE INVENTION

The present disclosure provides a method including the steps of identifying a compound dilution curve in an arterial vessel downstream of a pulmonary artery. The term “compound” reflects the recognition that a measured or identified dilution curve has a contributions from three components (i) passage of an indicator after passing through a single ventricle heart directly into the arterial vessel; (ii) passage of indicator after passing from the single ventricle heart to lungs, through the single ventricle heart and then to the arterial vessel; and (iii) passage of the indicator of components (i) and (ii) returning from systemic circulation and passing back into heart and to then the arterial vessel (wherein (i) and (ii) are referred to a first pass and (iii) is referred to as second pass).

In one configuration, a method is provided for assessing a ratio of pulmonary flow and a systemic flow, in a patient having a single ventricle heart, wherein the assessment corresponds to (i) a determined first component of a compound dilution curve measured in an arterial vessel downstream of a pulmonary artery, the first component of the compound dilution curve attributed to passage of an indicator after passing through the single ventricle directly into the arterial vessel and (ii) a determined second component of the compound dilution curve, the second component attributed to indicator after passing from the single ventricle heart to lungs, through the single ventricle heart and then to the arterial vessel.

It is understood the first component can be a first dilution curve and the second component can be a second dilution curve. Further, at least a portion of one of the first component and the second component can be modeled. The assessment of flows can be a ratio of areas under the first and second dilution curves, a height of the dilution curves or a characteristic of the first and second dilution curves. The shape of the first and second dilution curves can be estimated or modeled, wherein a portion of the curves can be estimated or modeled as one of, but not limited to, a linear, polynomial, exponential or logarithmic shape or segment.

It is also contemplated the shape of the third component of compound dilution curve can be estimated or modeled, wherein a portion of the third component curve can be estimated as, but not limited to at least one of linear, polynomial, exponential or logarithmic—thereby allowing for elimination of the influence of the third component on one or both of the first component and the second component. The indicator can be introduced into a venous vessel or the artia of the single ventricle heart.

A method is further provided for assessing a ratio of pulmonary flow and systemic flow in a patient having a single ventricle heart, by introducing an indicator into a venous vessel or artia of the single ventricle heart; identifying a compound dilution curve in an arterial vessel downstream of a pulmonary artery in a single ventricle heart cardiopulmonary system; attributing a first component of the compound dilution curve to passage of a first portion of the indicator after the first portion passes through the single ventricle heart directly to the arterial vessel; attributing a second component of the compound dilution curve to passage of the indicator after passing from the single ventricle heart to the lungs, through the single ventricle heart and to the arterial vessel; and determining a ratio of pulmonary flow (Qₚ) to systemic flow (Qₛ) based on the first and second component.

An apparatus is provided wherein the apparatus includes a dilution sensor operably coupled to an arterial vessel, the dilution sensor generating a signal series representing a compound dilution curve, the compound dilution curve including a first component representing passage of an indicator through a single ventricle heart and directly into the arterial vessel and a second component representing passage of an indicator through the single ventricle heart, through a pulmonary circuit, through the single ventricle heart and into the arterial vessel; and a controller connected to the dilution sensor, the controller configured to determine at least one characteristic of the first component and the second component and determine a ratio of a pulmonary flow to a systemic flow based on the determined at least one characteristic of the first component and the second component.

The controller can identify the first component as a first dilution curve as the second component as a second dilution curve. Further, the controller can determine the ratio of pulmonary flow to systemic flow corresponding to the first dilution curve and the second dilution curve.

### BRIEF DESCRIPTION OF THE SEVERAL VIEWS OF THE DRAWING(S)

FIG. 1 is a schematic representation of a single ventricle heart with pulmonary artery connected directly to the single ventricle through a shunt.

FIG. 2 is a schematic representation of a single ventricle heart with pulmonary artery connected to the single ventricle via an aorta and shunt, wherein Qₛ also includes coronary flow not shown in FIG. 2.

FIG. 3 is a graph showing the component dilution curves as would be identified at an arterial location, downstream of the pulmonary artery.

FIG. 4 is a graph showing a compound dilution curve, including the component dilution curves of FIG. 3, as identified at the arterial location, downstream of the pulmonary artery.

FIG. 5 is a schematic representation of the components of an extracorporeal circuit incorporating the present system.

### DETAILED DESCRIPTION OF THE INVENTION

The present disclosure provides a method and apparatus for assessing pulmonary and systemic blood flow in patients with a single ventricle physiology (SVP), and more particularly to determining, by dilution indicator methods, a ratio of pulmonary blood flow (Qₚ) to systemic blood flow (Qₛ) in patients with SVP.

For purposes of description, the single ventricle physiology (SVP) is understood to encompass those physiologies unable to create a circulation in series due to anatomical defects. That is, SVP includes a cardiac defect in which there is only one functioning ventricle; wherein the single ventricle may be a morphological right or left ventricle, or indeterminate; such as due to an underdeveloped chamber, valve, or outflow tract, or there may be two good-sized ventricles where the inflow and/or outflow tracts cannot be separated. Specific examples include hypoplastic ventricle (Hypoplastic Left Heart Syndrome); AV valve atresia (Tricuspid Atresia); abnormal inlet (Double inlet left ventricle) and inability to septate (Heterotaxy Syndrome with Left Atrial Isomerism, Double Outlet right Ventricle).

For cardiopulmonary and vascular systems, the term “upstream” of a given position refers to a direction against the flow of blood, and the term “downstream” of a given position is the direction of blood flowing away from the given position. The “arterial” side or portion is that part in which oxygenated blood flows from the heart to the capillaries. The “venous” side or portion is that part in which blood flows from the capillaries to the heart and lungs (the cardiopulmonary system).

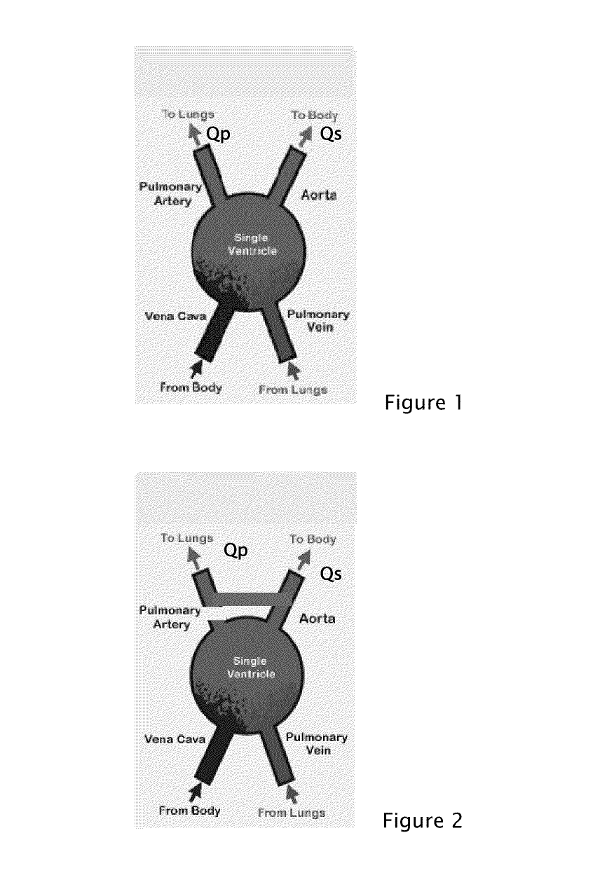

Referring to FIGS. 1 and 2, after intravenous injection of an indicator, that can be introduced into the vein or into the right atria (not shown), the introduced indicator will enter the ventricle (“From Body”). After mixing with blood from the lungs (From Lungs), the indicator will be pumped directly into the aorta and into the pulmonary artery PA (FIG. 1) or into the aorta and then to the PA (FIG. 2).

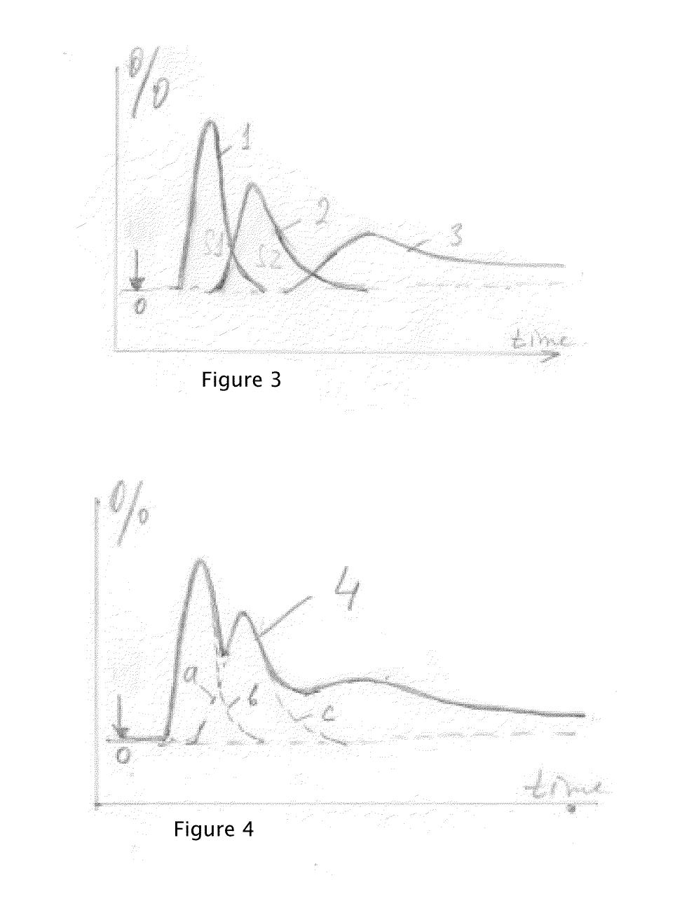

In a dilution curve recorded (identified or sample taken) at an arterial site in the arterial tree—the aorta or any peripheral artery, arteriolar, or any arterialized blood, the part of dilution curve that is produced from the “first pass” of the indicator, includes a first portion (FIG. 3, curve 1) and a second portion (FIG. 3 curve 2). That is, the compound dilution curve from the first pass includes a component from the indicator passing through the single ventricle heart directly to the arterial site and a component passing from the single ventricle heart, through the lungs, through the single ventricle heart again and then to the arterial site. Determining the components of the first pass from the total compound curve (FIG. 4) can include eliminating the influence of indicator that has already passed systemic circulation, including myocardial (the second pass) seen as curve 3 in FIG. 3.

The first portion of the first pass indicator (passing through the aorta into systemic circulation (to the body) will first reach the arterial recording (identifying) site in the arterial vessel, thereby resulting in the curve 1 shown in FIG. 3.

The second portion of the first pass indicator passes to the lungs, then from the lungs to again enter the single ventricle heart and then again divides into two parts. One part will go, via the aorta (the same route as first portion), to be recorded at the arterial site and start forming curve 2, as seen in FIG. 3 and the second part will go again back into the lungs, again the single ventricle heart and then into the aorta etc, until all first pass indicator will leave the heart and lungs and enter the systemic circulation.

From these configurations, the mass balance of the flows provides:

Qₛᵥ=Qₛ+Qₚ (Equation 1),

where Qₛᵥ is the total blood flow produced by the single ventricle heart (SVP), Qₛ is the systemic blood flow and Qₚ is the pulmonary blood flow.

According to the Stuart Hamilton Equation for a dilution curve recorded at an arterial site:

$${Q}_{SV} = \frac{V}{S 1} \qquad ( Equation 2 )$$

where V is the volume of the injected indicator and S1 is the area under the portion (component) of the total (compound) dilution curve (FIG. 4) formed by just the first portion of the first pass indicator as sensed at the arterial site, as shown in FIG. 3, as curve 1. That is, S1 is the area under the independent (component) curve resulting from the independent contribution of the indicator being sensed at the arterial site after passing from the heart directly to the arterial site, shown as curve 1 in FIG. 3. Thus, S1 in FIG. 3 is a component of the compound curve of FIG. 4.

$${Q}_{S} = \frac{V}{( S 1 + S 2 )} \qquad ( Equation 3 )$$

where S2 is the area under the portion (component) of the total (compound) dilution curve (FIG. 4) that is formed by the second portion of the first pass indicator as the second portion is sensed at the arterial site, as shown in FIG. 3 as curve 2 and (S1+S2) is the area under the first pass dilution curve. That is, S2 is the area under the curve resulting from the second portion of the indicator after passing from the single ventricle, through lungs, again through the single ventricle and then to the arterial sensing site. S2 is the component of the compound dilution curve of FIG. 4 that is attributable to this portion of the indicator.

Substituting V in Equation 2 from Equation 3 provides:

$${Q}_{SV} = {Q}_{S} \frac{( S 1 + S 2 )}{S 1} \qquad ( Equation 4 )$$

Substituting Qₛᵥ in Equation 1 from Equation 4 provides:

$$\begin{matrix}{Q}_{P} + {Q}_{S} = {Q}_{S} \frac{( S 1 + S 2 )}{S 1} & ( Equation 5 ) \\ {Q}_{P} + {Q}_{S} = {Q}_{S} ( 1 + \frac{S 2}{S 1} ) & ( Equation 5 a ) \\ {Q}_{P} + {Q}_{S} = {Q}_{S} + \frac{{Q}_{S} S 2}{S 1} & ( Equation 5 b ) \\ {Q}_{P} = \frac{{Q}_{S} S 2}{S 1} & ( Equation 6 ) \\ \frac{{Q}_{P}}{{Q}_{S }} = \frac{S 2}{S 1} & ( Equation 7 )\end{matrix}$$

Equation 7 suggests that in the case of SVP (as well as Norwood or analogous circulation, where one portion of the blood is pumped directly into systemic circulation and a second portion is pumped into the lungs by SVP), the assessment of pulmonary and systemic blood flow, such as a value of Qₚ/Qₛ, can be determined from the dilution curves, and in certain configurations, from the associated areas or parameters of the dilution curves.

However, in practice, the shape of the resulting dilution curve will also be influenced by systemic recirculation (curve 3 FIG. 3), such as a second pass of the indicator at the arterial measuring site. Second pass of the same indicator is indicator that re-circulated back into the heart again, after passing parts of systemic circulation like the brain, the kidneys, the myocardium etc. Curve 3 of FIG. 3 shows the sensed second pass of the indicator. Therefore, the actual dilution curve sensed (recorded) at arterial site will be the sum of all three curves, hence the compound dilution curve as seen as curve 4 in FIG. 4.

For purposes of description, the term compound dilution curve or compound curve is understood to mean a dilution curve at least partly defined by the contribution from different portions of the indicator travelling through different flow paths before simultaneously passing the sensor. For example, in the present description, the compound dilution curve includes a component from indicator passing directly from the SVP to the arterial sensor and a component from the indicator passing from the SVP, through the lungs, again through the SVP and then to the arterial sensor, wherein a portion of the components simultaneously pass the sensor.

As a further example, the compound curve can include a measured or identified dilution curve having contributions from three components (i) passage of an indicator after passing through a single ventricle heart directly into the arterial vessel; (ii) passage of indicator after passing from the single ventricle heart to lungs, through the single ventricle heart and then to the arterial vessel; and (iii) passage of the indicator of components (i) and (ii) returning from systemic circulation and passing back into the heart and to then the arterial vessel.

There are a number of processes or procedures to calculate or determine the components of the compound dilution curve or effectively extract or identify the components from the compound dilution curve. Specifically, one way to identify the components is to determine the curves 1 and 2 (and hence areas S1 and S2) from the compound dilution curve of FIG. 4.

In one mechanism, the shape of each curve 1 and 2 of FIG. 4, without contribution of the other, is estimated. Referring to FIG. 4, exemplary mechanisms for estimating or extracting the “hidden” sections of curve 1 and curve 2 from the compound curve include characterizing or modeling the shape of an upslope of curve 2 as linear (a); a down slope of curve 1 as an exponential (b); and a down slope of curve 2 as exponential (c).

It is also contemplated the shape of the third component (such as the second pass indicator) of compound dilution curve can be estimated or modeled, wherein a portion of the curve resulting from third component can be estimated as at least one of linear, polynomial, exponential or logarithmic. This estimation or modeling allows for the elimination of the influence of the third component (such as the second pass indicator) on one or both of the first component and the second component.

The chosen model for extraction, approximation or estimation of the component dilution curves may depend on the shape of recorded compound curve. Mathematical modeling of the compound dilution curve may use different mathematical functions to estimate the shape of component dilution curves, such as linear, logarithmic or polynomial of different power or other mathematical equations.

In addition, it is understood the value of Q_{P}/Qₛ can be estimated, based on or correspond to values other than the area under the respective areas of the extracted or estimated components of the compound dilution curve. For example, the height of the component curves 1 and 2, or the timing of the component curves, the duration of the component curves or other parameters of the component curves can be used as values related or proportional to the respective areas.

Alternatively, or additionally, to assist in identifying the component contributions to the compound dilution curve, such as S1 and S2, two or more indicators with different properties including but not limited to diffusion properties may be used. For example microspheres that do not pass the lung capillaries can be used. In this case, the dilution curve from intravenous injection of microspheres will only exhibit the first portion of indicator at the arterial sampling site. This dilution curve in combination with curve FIG. 4 from indicator that passes lungs can help to identify curve 2, for example by subtracting one curve from other. The use of two indicators includes, but is not limited to indicators with different diffusible properties.

To generate a dilution curve, any change in the physical or chemical blood property can be sensed, identified or measured. These physical properties include, but not limited to thermal properties, optical properties, electromagnetic properties, blood density and others. Blood concentration of ions, gas concentrations, protein concentrations, radio isotopes and other concentration changes can be introduced in the blood to generate dilution curves, and hence the compound dilution curve. The dilution curves may be expressed in % concentration units or no units or in other units like volts or in units of blood parameters that were changed or in units of substances that were injected like isotopes and others.

Thus, the indicator includes but is not limited to: blood hematocrit, blood protein, sodium chloride, dyes, blood urea nitrogen, a change in ultrafiltration rate, glucose, lithium chloride and radioactive isotopes and microspheres, or any other measurable blood property or parameter. An injectable indicator may be any of the known indicators including saline, electrolytes, water and temperature gradient indicator bolus. Preferably, the indicator is non toxic with respect to the patient and non reactive with the material of the system. The indicator may be any substance that will change a blood chemical or physical characteristic. The indicator may be a physically injected material such as saline. Alternatively, the indicator may be by manipulating blood properties without introduction of an indicator volume, such as by heating or cooling the blood or changing electromagnetic blood properties or chemical blood properties.

A sensor is employed to detect passage of the indicator and thus measures, identified or monitors a blood parameter or property, and particularly variations of the blood parameter or property. Thus, the sensor is capable of sensing a change is a blood property, parameter or characteristic. For purposes of the disclosure, the sensor can be referred to as a dilution sensor, but this label is not intended to limit the scope of available sensors. Ultrasound velocity sensors as well as temperature sensors and optical sensors, density or electrical impedance sensors, chemical or physical sensors may be used to detect changes in blood parameters. It is understood that other sensors that can detect blood property changes may be employed. The operating parameters of the particular system will substantially dictate the specific design characteristics of the dilution sensor, such as the particular sound velocity sensor. For example, if a thermal sensor is employed, the thermal sensor can be any sensor that can measure temperature, for example, but not limited to thermistor, thermocouple, electrical impedance sensor (electrical impedance of blood changes with temperature change), ultrasound velocity sensor (blood ultrasound velocity changes with temperature), blood density sensor and analogous devices. Therefore, any type of optical sensor, impedance, resistance or electrical sensors which measure a changeable blood parameter such as the sound or ultrasound velocity in blood can be calibrated. Electrical resistance of the blood can be measured, as the resistance depends on the volume of red blood cells (hematocrit). Calibration can be provided for ultrasound velocity sensors, as well as temperature sensors and optical density, density or electrical impedance sensors can be used to detect changes in blood parameters.

It is understood the sensor or sensors for recording the compound dilution curve from the changes in any physical or any chemical blood property can be located in the blood vessels including the heart—using different type of catheters like for example thermodilution catheters.

Further with respect to the sensor, the sensor can be located around or attached to blood vessels or the heart for example, such as perivascular sensors that use ultrasound waves or electrical waves to measure blood properties. The sensors can be located on the surface of the body, for example, sensing blood optical property changes on fingers. It is further understood the sensor may be located outside the body for example analyzing blood that withdrawn from the vessels such as ultrasound dilution sensors or lithium dilution sensors. Further, sensing blood properties outside the body can be performed through the optical line (fiber optics) located in the blood vessel; or sensing blood propertied through electromagnetic waves by sensors located above the body for example magnetic resonance systems or for example fluorescent indicator sensing systems. Thus, the dilution sensor can be operably coupled to the vessel either through physical contact with the vessel, being inside the vessel, being outside the vessel or spaced from the vessel.

In one example, newborns that suffer single ventricle pathology (SVP) are typically small weight patients and there will be a problem to insert specialized dilution catheters inside tiny blood vessels. In one configuration, ultrasound dilution technology can be used, which does not require insertion of specialized catheters, but can use existing catheters to perform dilution measurements. The access to arterial blood is achieved by withdrawing blood through existing catheters and delivering it back through venous catheter. These catheters are routinely available during and after the surgery in this patent population.

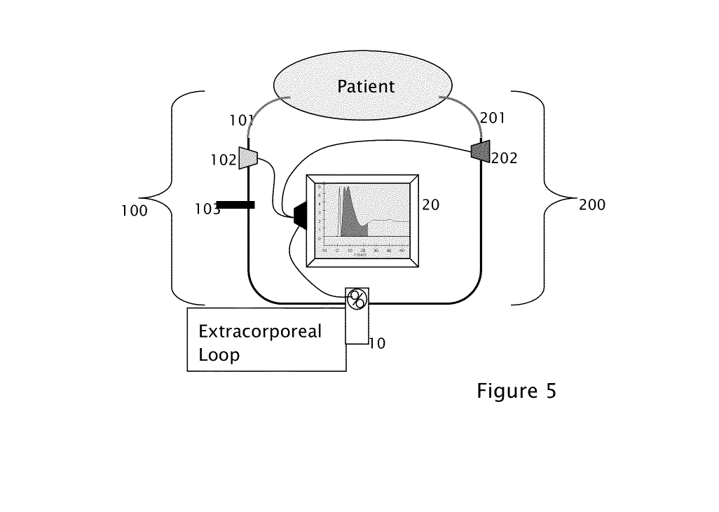

FIG. 5 represents one configuration of the system in terms of an extracorporeal circuit or loop. The extracorporeal circuit includes a venous line 100, a venous catheter 101, a venous sensor 102, an injection site 103, a pump 10, an arterial line 200, an arterial catheter 201 and an arterial sensor 202 in the arterial line, wherein a controller 20 is connected to the arterial sensor 202 and typically to the pump.

The pump 10 can be any of a variety of pumps types, including but not limited to a roller (or impeller) pump. The pump 10 induces a blood flow through the extracorporeal circuit. At least one of the pump 10 and the controller 20 typically include control of the pump and the flow rate of the blood through the pump. The pump 10 can be at any of a variety of locations in the extracorporeal circuit, and is not limited to the position shown in FIG. 5.

In this configuration, the arterial sensor 202 is a dilution sensor that measures ultrasound velocity of the blood. The indicator is body temperature isotonic saline. The ultrasound velocity in blood (1560-1590 m/sec) is primarily a function of the total blood protein and ion concentrations. Ultrasound velocity in body temperature isotonic saline is approximately 1533 m/sec. Thus, an intravenous bolus administration of isotonic saline causes a decrease in ultrasound velocity, as the indicator dilutes the blood.

The arterial side 200 of the extracorporeal circuit connects to the arterial catheter (that usually already exists in the patient, or can be inserted) for blood withdrawal. It is understood the arterial catheter can be located in the femoral, carotid, or radial arteries, or any other artery including the aorta.

The venous line 100 of the extracorporeal circuit, for blood delivery, connects to the venous catheter 101 with the catheter tip usually located in a vein or in the right atria. The venous line 100 also provides an injection site 103 for the introduction of the dilution indicator(s), such as the previously recited body temperature isotonic saline.

The controller 20 is operably connected to the pump 10 and the sensors 102, 202. The controller 20 can be a stand alone device connected to a computer, or a dedicated device such as a flowmeter or monitor with an onboard computer. It is understood the controller 20 and the flowmeter can be integrated into a single unit, and thus function as a monitor. In this arrangement, the flowmeter can be an HD02 flowmeter manufactured by Transonic Systems Inc. of Ithaca N.Y. The HD02 Flowmeter comes with standard software to interface with the standard personnel computer available from Dell, HP etc. Other configurations of the sensors, flowmeter and controller are possible, such as combining the computer into the flowmeter as in the Transonic HCM101 meter or equivalent. The controller 20 is programmed to extract or estimate the components of the compound dilution curve and provide the relationship of Qₚ and Qₛ as set forth above.

In operation, it is contemplated that during the measurement procedure, blood is circulated out of the patient via the arterial line 200 and through the extracorporeal circuit by the pump 10 and back into patient via the venous line 100. In one configuration, an injection of body temperature isotonic saline is performed into injection port 103. However, it is understood the introduction of the indicator can be into a venous vessel or the atria of the single ventricle heart. That is, the indicator is introduced on the venous side of the cardiopulmonary system or extracorporeal loop upstream of the ventricle of the single ventricle heart. The indicator therefore passes from the single ventricle heart after having at least mixed with blood in the single ventricle.

After intravenous injection of the indicator, the indicator, saline, will pass the venous sensor 102 which will start or trigger a portion of the software in the controller 20. The indicator will pass the single ventricle (single ventricle heart). From there after mixing with blood coming from the lungs, the first portion of indicator will be pumped into to the systemic circulation of the patient via the aorta, while the second portion of the indicator will be pumped into to the lungs via the PA, as seen in FIGS. 1 and 2. The arterial sensor 202 detects the compound dilution curve including the first component related to the first portion of indicator and then the second portion of indicator after it multiple times circulated in cardiopulmonary system (FIG. 4). The compound dilution curve will also include contribution from the part of the indicator passing from systemic recirculation into the heart after the indicator has passed parts of systemic circulation like brain, kidney myocardium etc. The resulting compound dilution curve will be analyzed in the controller 20 to determine the Qₚ/Qₛ values based on Equation 7, or similarly derived equations and the chosen algorithm for estimating or extracting estimations of the individual components, such as areas S1 and S2 or related values under the estimated dilution curves 1 and 2, respectively.

The invention has been described in detail with particular reference to a presently preferred embodiment, but it will be understood that variations and modifications can be effected within the spirit and scope of the invention. The presently disclosed embodiments are therefore considered in all respects to be illustrative and not restrictive. The scope of the invention is indicated by the appended claims, and all changes that come within the meaning and range of equivalents thereof are intended to be embraced therein.

1. A method comprising:
(a) identifying a compound dilution curve in an arterial vessel downstream of a pulmonary artery, the compound dilution curve having a contributions from (i) passage of an indicator after passing from a single ventricle heart directly into the arterial vessel and (ii) passage of indicator after passing from the single ventricle heart to lungs, through the single ventricle heart and then to the arterial vessel;
b) attributing (i) a first component of the compound dilution curve to the indicator after passing through the single ventricle heart directly into the arterial vessel and (ii) a second component of the compound dilution curve to the indicator after passing from the single ventricle heart to the lungs, through the single ventricle heart and then to the arterial vessel; and
(c) assessing a pulmonary flow and a systemic flow ratio corresponding to the attributed first component and second component.

2. The method of claim 1 wherein the first component is a first dilution curve and the second component is a second dilution curve.

3. The method of claim 2, wherein at least a portion of one of the first dilution curve and the second dilution curve is modeled.

4. The method of claim 2, wherein the ratio of pulmonary flow (Qₚ) to systemic flow (Qₛ) corresponds to S2 to S1, where S2 is the area under the second dilution curve and S1 is the area under the first dilution curve.

5. The method of claim 2, wherein at least a portion of at least one of the first dilution curve and the second dilution curve is one of linear, polynomial, logarithmic or exponential.

6. The method of claim 2, wherein assessing the pulmonary flow and the systemic flow corresponds to one of a height, a time, an area or a characteristic of the first dilution curve and the second dilution curve.

7. A method comprising:
(a) assessing a relation of a pulmonary flow and a systemic flow, in a patient having a single ventricle heart, the relation corresponding to (i) a determined first component of a compound dilution curve measured in an arterial vessel downstream of a pulmonary artery, the first component of the compound dilution curve attributed to passage of an indicator after passing from the single ventricle directly into the arterial vessel and (ii) a determined second component of the compound dilution curve, the second component attributed to indicator after passing from the single ventricle heart to lungs, through the single ventricle heart and then to the arterial vessel.

8. The method of claim 7, wherein the first component is a first dilution curve and the second component is a second dilution curve.

9. The method of claim 8, wherein at least a portion of one of the first dilution curve and the second dilution curve is modeled.

10. The method of claim 8, wherein the ratio of pulmonary flow (Qₚ) to systemic flow (Qₛ) corresponds to S2 to S1, where S2 is the area under the second dilution curve and S1 is the area under the first dilution curve.

11. The method of claim 8, wherein at least a portion of at least one of the first dilution curve and the second dilution curve is one of linear, polynomial, logarithmic or exponential.

12. The method of claim 8, wherein assessing the pulmonary flow and the systemic flow corresponds to one of a height, a time, an area or a characteristic of the first dilution curve and the second dilution curve.

13. A method comprising:
(a) assessing a ratio of a pulmonary flow and a systemic flow, in a patient having a single ventricle heart, based on (i) a first component of a compound dilution curve in an arterial vessel downstream of a pulmonary artery, the first component attributed to passage of a first portion of an indicator after passing from the single ventricle heart and directly to the arterial vessel and (ii) a second component of the compound dilution curve attributed to a second portion of the indicator after the second portion passing through the single ventricle heart then to the lungs, through the single ventricle heart and to the arterial vessel.

14. The method of claim 13, wherein assessing the pulmonary flow and the systemic flow includes determining a ratio of pulmonary flow to systemic flow.

15. A method comprising:
(a) introducing an indicator upstream of a ventricle in a single ventricle heart cardiopulmonary system;
(b) identifying a compound dilution curve in an arterial vessel downstream of a pulmonary artery in the single ventricle heart cardiopulmonary system;
(c) attributing a first component of the compound dilution curve to passage of a first portion of the indicator after the first portion from through the single ventricle heart directly to the arterial vessel;
(d) attributing a second component of the compound dilution curve to passage of the indicator after passing from the single ventricle heart to the lungs, through the single ventricle heart and to the arterial vessel; and
(e) determining a ratio of pulmonary flow (Qₚ) to systemic flow (Qₛ) based on the first and second component.

16. A method comprising:
(a) obtaining, in a patient having a single ventricle heart, a dilution curve in an arterial vessel downstream of a pulmonary artery, the dilution curve having a contribution from at least (i) passage of a first portion of an indicator after passing through the single ventricle heart and directly to the arterial vessel and (ii) passage of a second portion of the indicator after the second portion passing through the single ventricle heart then to the lungs, through the single ventricle heart and to the arterial vessel;
(b) modeling at least a portion of a dilution curve attributable to one of the first portion and the second portion of the indicator; and
(c) determining a ratio of the pulmonary flow to the systemic flow at least partly based on the modeled portion of the dilution curve.

17. An apparatus comprising:
(a) a dilution sensor operably coupled to an arterial vessel, the dilution sensor generating a signal series representing a compound dilution curve, the compound dilution curve including a first component representing passage of an indicator through a single ventricle heart and directly into the arterial vessel and a second component representing passage of an indicator through the single ventricle heart, through a pulmonary circuit, through the single ventricle heart and into the arterial vessel; and
(b) a controller connected to the dilution sensor, the controller configured to determine at least one characteristic of the first component and the second component and determine a ratio of a pulmonary flow to a systemic flow based on the determined at least one characteristic of the first component and the second component.

18. The apparatus of claim 17, wherein the controller determines a first dilution curve as the first component and a second dilution curve as the second component.

19. The apparatus of claim 17, wherein the first component is a first dilution curve and the second component is a second dilution curve, and the ratio of the pulmonary flow to the systemic flow corresponds to one of a height of the first and the second dilution curves, a time of the first and the second dilution curves, an area under the first and the second dilution curves or a duration of a given portion of the first and the second dilution curves.

20. The apparatus of claim 17, wherein the controller determines the ratio of pulmonary flow to systemic flow corresponding to the first dilution curve and the second dilution curve.
